(19) United States
(12) Plant Patent Application Publication
(10) Pub. No.: US 2017/0094886 P1
(43) Pub. Date: Mar. 30, 2017

(54) Echinacea plant named 'Sweet271'

(50) ;

(71) Applicant: Henricus Maria Joseph Holtmaat, Zuidwolde (NL)

(72) Inventor: Henricus Maria Joseph Holtmaat, Zuidwolde (NL)

(73) Assignee: AB KWEKERSRECHTEN B.V., Zuidwolde (NL)

(21) Appl. No.: 14/756,668

(22) Filed: Sep. 29, 2015

(51) Int. Cl. A01H 5/00

(52) U.S. Cl. PLT/428

(57) ABSTRACT

A new and distinct cultivar of Echinacea plant named ‘Sweet271’, characterized by its upright and relatively compact plant habit; moderately vigorous growth habit; freely branching habit; strong and relatively short flowering stems; numerous anemone-centered inflorescences with red purple-colored florets; and good garden performance.

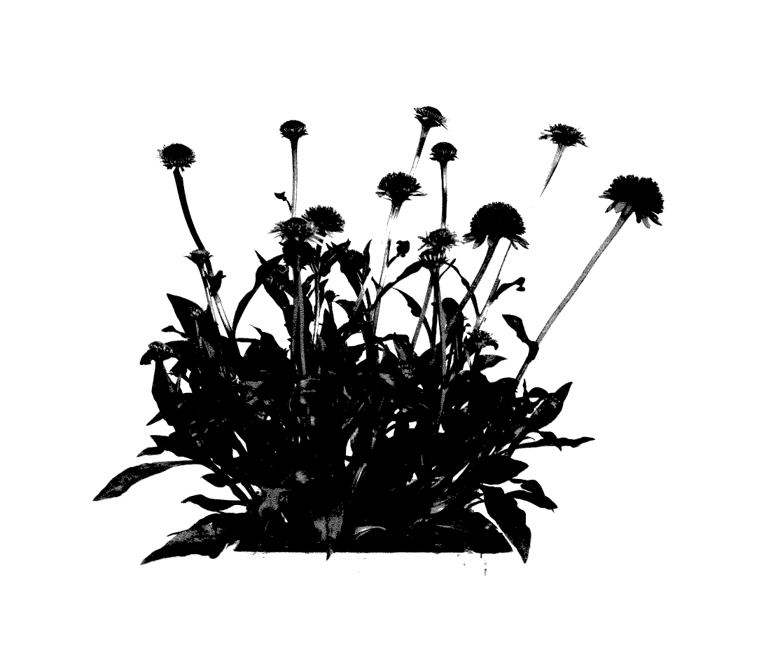

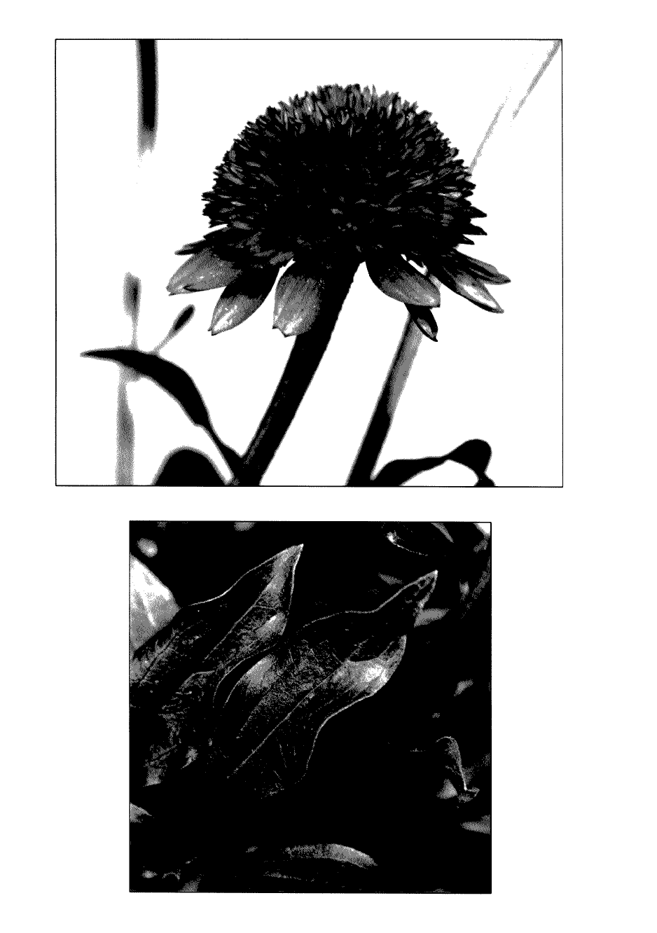

Botanical designation: Echinacea purpurea

Cultivar denomination: ‘SWEET271’

### BACKGROUND OF THE INVENTION

The present invention relates to a new and distinct cultivar of Echinacea plant, botanically known as Echinacea purpurea, and hereinafter referred to by the name ‘Sweet271’.

The new Echinacea plant is a product of a planned breeding program conducted by the Inventor in Zuidwolde, The Netherlands. The objective of the breeding program is to develop new compact and freely flowering Echinacea plants with unique and attractive ray floret coloration.

The new Echinacea plant originated from an open-pollination in July, 2011 in Zuidwolde, The Netherlands of a proprietary selection of Echinacea purpurea identified as code number 7067-05, not patented, as the female, or seed, parent with an unknown selection of Echinacea purpurea as the male, or pollen, parent. The new Echinacea plant was discovered and selected by the Inventor as a single flowering plant from within the progeny of the stated open-pollination grown in a controlled greenhouse environment in Zuidwolde, The Netherlands in June, 2013.

Asexual reproduction of the new Echinacea plant by tissue culture in a controlled environment in Zuidwolde, The Netherlands since January, 2014 has shown that the unique features of this new Echinacea plant are stable and reproduced true to type in successive generations.

### SUMMARY OF THE INVENTION

Plants of the new Echinacea have been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Sweet271’. These characteristics in combination distinguish ‘Sweet271’ as a new and distinct Echinacea plant:
1. Upright and relatively compact plant habit.
2. Moderately vigorous growth habit.
3. Freely branching habit.
4. Strong and relatively short flowering stems.
5. Numerous anemone-centered inflorescences with red purple-colored florets.
6. Good garden performance.

Plants of the new Echinacea can be compared to plants of the female parent selection. Plants of the new Echinacea differ from plants of the female parent in the following characteristics:
1. Plants of the new Echinacea have shorter leaves than plants of the female parent selection.
2. Plants of the new Echinacea and the female parent selection differ in floret color as plants of the female parent selection have yellow and red-colored florets.

Plants of the new Echinacea can be compared to plants of Echinacea purpurea ‘Piccolino’, disclosed in U.S. Plant Pat. No. 23,348. In side-by-side comparisons conducted in Zuidwolde, The Netherlands, plants of the new Echinacea differed primarily from plants of ‘Piccolino’ in the following characteristics:
1. Plants of the new Echinacea had darker green-colored leaves than plants of ‘Piccolino’.
2. Plants of the new Echinacea have darker red purple-colored florets than plants of ‘Piccolino’.

### BRIEF DESCRIPTION OF THE PHOTOGRAPHS

The accompanying photographs illustrate the overall appearance of the new Echinacea plant showing the colors as true as it is reasonably possible to obtain in colored reproductions of this type. Colors in the photographs may differ slightly from the color values cited in the detailed botanical description which accurately describe the colors of the new Echinacea plant. The photograph on the first sheet is a side perspective view of a typical flowering plant of ‘Sweet271’ grown in ground beds in an outdoor nursery and placed in a 17.5 cm by 22 cm container for the photograph. The photographs on the second sheet are close-up views of a typical inflorescence (upper photograph) and the upper surface of typical leaves (lower photograph) of ‘Sweet271’.

### DETAILED BOTANICAL DESCRIPTION

The aforementioned photographs and following observations and measurements describe plants grown during the summer in ground beds in an outdoor nursery in Zuidwolde, The Netherlands and under cultural practices typically used in commercial Echinacea production. During the production ofthe plants, day temperatures ranged from 18° C. to 30° C. and night temperatures ranged from 8° C. to 20° C. Plants were eight months old when the photographs and description were taken. In the following description, color references are made to The Royal Horticultural Society Colour Chart, 2007 Edition, except where general terms of ordinary dictionary significance are used.
Botanical classification: Echinacea purpurea ‘Sweet271’.
Parentage:
   Female parent.—Proprietary selection of Echinacea purpurea identified as code number 7067-05, not patented.
   Male parent.—Unknown selection of Echinacea purpurea, not patented.
Propagation:
   Type.—By tissue culture.
   Time to initiate roots, summer.—About one week at temperatures about 25° C.
   Time to produce a rooted young plant, summer.—About five weeks at temperatures about 21° C.
   Root description.—Fine, fibrous; typically pale cream in color, actual color of the roots is dependent on substrate composition, water quality, fertilizer type and formulation, substrate temperature and physiological age of roots.
   Rooting habit.—Freely branching; medium density.
Plant description:
   Plant form and growth habit.—Herbaceous perennial; upright and relatively compact plant habit, broad inverted triangle; freely branching habit with about 20 lateral branches developing per plant; moderately vigorous growth habit.
   Plant height.—About 40.5 cm.
   Plant diameter or spread.—About 38.1 cm.
   Lateral branches.—Length: About 24.3 cm. Diameter: About 4 mm. Internode length: About 4.5 cm. Aspect: Erect to about 25° from vertical. Strength: Strong. Texture: Smooth, glabrous. Color: Close to 144A to 144B tinged with close to 175B.
Leaf description:
   Arrangement.—Alternate, simple.
   Length.—About 8.3 cm.
   Width.—About 3 cm.
   Shape.—Narrowly ovate to narrowly elliptic.
   Apex.—Acute.
   Base.—Attenuate.
   Margin.—Entire to very shallowly dentate; undulate.
   Texture, upper and lower surfaces.—Moderately pubescent, strigose; rough.
   Luster, upper and lower surfaces.—Slightly glossy.
   Venation pattern.—Pinnate.
   Color.—Developing leaves, upper surface: Close to 143A. Developing leaves, lower surface: Close to 146A to 146B. Fully expanded leaves, upper surface: Close to N137A; venation, close to 145A. Fully expanded leaves, lower surface: Close to 137B; venation, close to 145A.
   Petioles, basal leaves.—Length: About 2.8 cm. Diameter: About 3 mm by 4 mm. Strength: Strong, flexible. Texture, upper surfaces: Mostly glabrous; margins, moderately pubescent, strigose. Color, upper surface: Close to 145A; margins, close to 141A. Color, lower surface: Close to 145A; margins, close to 13 7A.
Inflorescence description:
   Appearance.—Anemone-centered inflorescences with ray and disc florets arranged on a capitulum; inflorescences positioned upright above the foliar plane on mostly upright and strong peduncles.
   Flowering habit.—Freely flowering habit with about 60 inflorescences developing per plant during the flowering season.
   Fragrance.—None detected.
   Time to flower.—In the garden, plants flower continuously from early July to late September in The Netherlands.
   Inflorescence longevity.—Inflorescences maintain good substance for about three weeks on the plant; inflorescences persistent.
   Inflorescence buds.—Height: About 1.4 cm. Diameter: About 3.4 cm. Shape: Flattened globular. Color: Involucral bracts, close to 150B; ray florets, close to 182D with apices, close to 151D.
   Inflorescence size.—Diameter: About 5.5 cm. Depth (height): About 3.6 cm. Disc diameter: About 4.7 cm. Receptacle height: About 9 mm. Receptacle diameter: About 9 mm. Receptacle color: Close to 158A; edges, close to 64D.
   Ray florets.—Quantity and arrangement: About twelve arranged in a single whorl at the base of the receptacle. Length: About 2.4 cm. Width: About 8 mm. Shape: Oblanceolate. Apex: Emarginate to praemorse. Base: Cuneate. Margin: Entire. Texture, upper surface: Glabrous; velvety; slightly longitudinally ridged. Texture, lower surface: Glabrous; longitudinally ridged. Luster, upper surface: Matte. Luster, lower surface: Moderately glossy. Aspect: Horizontal to drooping, about 30° from horizontal. Color: When opening, upper surface: Close to between 61A and 67A. When opening, lower surface: Close to 59D. Fully opened, upper surface: Close to 61A; color becoming closer to 61C with development. Fully opened, lower surface: Close to 58D; color does not change with development.
   Disc florets.—Quantity and arrangement: About 150 per inflorescence, spirally at the center. Length: About 1.8 cm. Diameter: About 6 mm. Shape: Obovate. Apex: Praemorse. Base: Cuneate, fused. Margin: Entire. Texture, upper surface: Smooth, glabrous; velvety. Texture, lower surface: Smooth, glabrous. Luster, upper surface: Matte. Luster, lower surface: Slightly glossy. Color: When opening, upper surface: Close to 63A. When opening, lower surface: Close to 60C. Fully opened, upper surface: Close to 61B; color does not change with development. Fully opened, lower surface: Close to 59D; color does not change with development.
   Receptacle spines.—Quantity: One per disc floret. Shape: Acicular. Apex: Acute. Base: Attenuate. Texture: Smooth, glabrous. Luster: Glossy. Color: Apex: Close to 42A to 42B. Mid-section: Close to 144A to 144B. Base: Close to 157D.
   Involucral bracts.—Quantity per inflorescence: About 44 arranged in about two whorls. Length: About 8 mm. Width: About 3 mm. Shape: Ovate. Apex: Acute. Base: Cuneate. Margin: Entire. Texture, upper and lower surfaces: Mostly glabrous; margins, moderately pubescent. Luster, upper and lower surfaces: Matte. Color, upper surface: Close to N137B. Color, lower surface: Close to 137A.
   Peduncles.—Length: About 12.4 cm. Diameter: About 4 mm. Strength: Strong. Aspect: Upright to about 25° from vertical. Texture: Smooth, glabrous. Color: Close to 144A to 144B.
   Reproductive organs.—Androecium: None observed on ray or disc florets. Gynoecium (present only on disc florets): Quantity per floret: One. Pistil length: About 6 mm. Stigma shape: Decurrent, unequal. Stigma color: Close to between 187A and 200A. Style length: About 4.5 mm. Style color: Close to 59D. Ovary color: Close to 157D. Seeds and fruits: Seed and fruit development have not been observed on plants of the new Echinacea.
Disease & pest resistance: Plants of the new Echinacea have not been shown to be resistant to pathogens and pests common to Echinacea plants.
Garden performance: Plants of the new Echinacea have exhibited good garden performance and to tolerate rain and wind. Plants of the new Echinacea have been observed to tolerate high temperatures of about 35° C. and to be hardy to USDA Hardiness Zones 3 to 4.

It is claimed:

1. A new and distinct Echinacea plant named ‘Sweet271’ as illustrated and described.
